(19) United States
(12) Patent Application Publication
(10) Pub. No.: US 2025/0269165 A1
(43) Pub. Date: Aug. 28, 2025

(54) TRANSFER SET WITH INTEGRATED DISINFECTANT

(71) Applicants: BAXTER INTERNATIONAL INC., Deerfield, IL (US); BAXTER HEALTHCARE SA, Glattpark (Opfikon) (CH)

(72) Inventor: Brian James Connell, Evanston, IL (US)

(21) Appl. No.: 18/585,689

(22) Filed: Feb. 23, 2024

(51) Int. Cl. A61M 39/16; A61M 1/28; A61M 39/10

(52) U.S. Cl. CPC A61M 39/162; A61M 1/285; A61M 2039/1038

(57) ABSTRACT

A transfer set with integrated disinfectant includes a main body, a cap, and a disinfectant chamber. The main body has a hollow tube shape, a catheter end, and a connecting end that includes mating components. The cap is configured to engage with the mating components. The disinfectant chamber is integrated around an outer circumference of the main body. The catheter end selectively couples to tubing. The disinfectant chamber is located between the catheter end and the mating components. The disinfectant chamber includes a closed end adjacent the catheter end and an open end adjacent the mating components. The disinfectant chamber contains a disinfectant.

### FIELD

The present disclosure relates generally to a transfer set with an integrated disinfectant chamber. More specifically, this disclosure relates to a transfer set with an integrated disinfectant chamber that diffuses disinfectant through a gap, to disinfect transfer set threads.

### BACKGROUND

Bacterial contamination is a large concern for patients undergoing medical treatments, such as peritoneal dialysis. For example, introducing bacteria into the peritoneal cavity could lead to peritonitis, which can endanger the patient's life. Bacteria is often introduced via touch contamination, such as when handling transfer sets. The current method to protect patients from inadvertently infusing bacteria via a potential touch contamination is the use of disinfectant disconnect caps. A disinfectant disconnect cap typically has a povidone-iodine solution disposed on a sponge within the cap itself. When patients complete their treatment, they disconnect a cycler set or bag from their transfer set, and subsequently screw on a new disconnect cap onto their transfer set. The povidone-iodine solution from the sponge in the cap coats the transfer set threads, and disinfects the thread surfaces, to eliminate bacterial load and reduce contamination risk during subsequent connections. However, the use of disinfectant disconnect caps has multiple drawbacks. First, the povidone-iodine solution is aqueous-based and is usually protected within a foil pouch. Also, a new disconnect cap must be used after each transfer, which is costly and produces undesirable waste. Furthermore, disconnect caps typically have a shelf life; unused disconnect caps must be discarded. It should be appreciated that the difficulties with touch contamination, and related disinfection, are applicable to many different connection interfaces in the medical space, beyond the peritoneal dialysis example provided above.

A transfer set with integrated disinfectant chamber that is self-contained and resealable is therefore needed.

### SUMMARY

The transfer set with integrated disinfectant disclosed herein improves on current transfer set disinfection technology by integrating the disinfectant within the transfer set and diffusing the disinfectant directly onto the connector, so as to sterilize the transfer set connector between transfers. This integrated disinfectant advantageously reduces the need for using external disinfectant sources between transfers, thus ensuring consistent connector-thread disinfection and reducing related production costs and waste.

In light of the disclosure herein, and without limiting the scope of the invention in any way, in a first aspect of the present disclosure, which may be combined with any other aspect listed herein unless specified otherwise, a transfer set with integrated disinfectant includes a main body, a cap, and a disinfectant chamber. The main body includes a hollow tube shape, a catheter end, and a connecting end that includes mating components. The cap is configured to engage with the mating components. The disinfectant chamber is integrated around an outer circumference of the main body. The catheter end selectively couples to tubing. The disinfectant chamber is located between the catheter end and the mating components. The disinfectant chamber includes a closed end adjacent the catheter end and an open end adjacent the mating components. The disinfectant chamber contains a disinfectant.

In a second aspect of the present disclosure, which may be combined with any other aspect listed herein unless specified otherwise, the transfer set further includes a barrier located between the disinfectant chamber and the mating components, such that the barrier covers the open end and prevents the disinfectant from flowing towards the mating components.

In a third aspect of the present disclosure, which may be combined with any other aspect listed herein unless specified otherwise, the barrier is comprised of an impermeable, non-porous material.

In a fourth aspect of the present disclosure, which may be combined with any other aspect listed herein unless specified otherwise, the barrier is an O-ring.

In a fifth aspect of the present disclosure, which may be combined with any other aspect listed herein unless specified otherwise, the disinfectant is elemental iodine.

In a sixth aspect of the present disclosure, which may be combined with any other aspect listed herein unless specified otherwise, the disinfectant is an alcohol-based disinfectant.

In a seventh aspect of the present disclosure, which may be combined with any other aspect listed herein unless specified otherwise, the barrier is comprised of one of a fluoro compound or a fluoro polymer.

In an eighth aspect of the present disclosure, which may be combined with any other aspect listed herein unless specified otherwise, the mating components are threaded.

In a ninth aspect of the present disclosure, which may be combined with any other aspect listed herein unless specified otherwise, the main body further includes at least one notched portion around the outer circumference of the main body, such that when the barrier covers the open end, there is at least one gap between the barrier and the open end of the disinfectant chamber so as to permit flow of disinfectant through the distance between the barrier and the open end of the disinfectant chamber.

In a tenth aspect of the present disclosure, which may be combined with any other aspect listed herein unless specified otherwise, a connector on a dialysis fluid bag obstructs the at least one gap between the barrier and the open end of the disinfectant chamber, such that the disinfectant remains in the disinfectant chamber.

In an eleventh aspect of the present disclosure, which may be combined with any other aspect listed herein unless specified otherwise, the cap further comprises a member configured to engage with an internal portion of the open end to seal the open end, such that the member prevents flow of the disinfectant.

In a twelfth aspect of the present disclosure, which may be combined with any other aspect listed herein unless specified otherwise, a transfer set with integrated disinfectant includes a main body, a releasable connector, and a disinfectant chamber. The main body includes a hollow tube shape, a catheter end, and a connecting end that includes mating components. The releasable connector is configured to engage with the mating components. The disinfectant chamber is integrated around an outer circumference of the main body. The catheter end selectively couples to tubing and the connecting end selectively couples to the releasable connector. The releasable connector includes a first end configured to engage with the mating components and a second end with a resealable port. The disinfectant chamber is located between the catheter end and the mating components. The disinfectant chamber includes a closed end adjacent the catheter end and an open end adjacent the mating components. The disinfectant chamber contains a disinfectant.

In a thirteenth aspect of the present disclosure, which may be combined with any other aspect listed herein unless specified otherwise, the transfer set further includes a barrier located between the disinfectant chamber and the mating components, such that the barrier covers the open end and prevents the disinfectant from flowing towards the mating components.

In a fourteenth aspect of the present disclosure, which may be combined with any other aspect listed herein unless specified otherwise, the barrier is comprised of an impermeable, non-porous material.

In a fifteenth aspect of the present disclosure, which may be combined with any other aspect listed herein unless specified otherwise, the disinfectant is one of elemental iodine or an alcohol-based disinfectant.

In a sixteenth aspect of the present disclosure, which may be combined with any other aspect listed herein unless specified otherwise, the mating components are threaded.

In a seventeenth aspect of the present disclosure, which may be combined with any other aspect listed herein unless specified otherwise, the main body further includes at least one notched portion around the outer circumference of the main body, such that when the barrier covers the open end, there is at least one gap between the barrier and the open end of the disinfectant chamber so as to permit flow of disinfectant through the distance between the barrier and the open end of the disinfectant chamber.

In an eighteenth aspect of the present disclosure, which may be combined with any other aspect listed herein unless specified otherwise, the first end of the releasable connector obstructs the at least one gap between the barrier and the open end of the disinfectant chamber, such that the disinfectant remains in the disinfectant chamber.

In a nineteenth aspect of the present disclosure, which may be combined with any other aspect listed herein unless specified otherwise, the resealable port on the second end of the releasable connector is configured to selectively fluidly couple to a connector on a dialysis fluid bag.

In a twentieth aspect of the present disclosure, which may be combined with any other aspect listed herein unless specified otherwise, the resealable port is configured to reseal after uncoupling to a connector on a dialysis bag.

Additional features and advantages of the disclosed devices, systems, and methods are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein. Moreover, it should be noted that the language used in the specification has been selected for readability and instructional purposes, and not to limit the scope of the inventive subject matter.

### BRIEF DESCRIPTION OF THE FIGURES

Understanding that figures depict only typical embodiments of the invention and are not to be considered to be limiting the scope of the present disclosure, the present disclosure is described and explained with additional specificity and detail through the use of the accompanying figures. The figures are listed below.

FIG. 1 illustrates a perspective view of a transfer set with integrated disinfectant chamber and a transfer cap, according to an example embodiment of the present disclosure.

FIG. 2 illustrates a perspective view of a transfer set with integrated disinfectant chamber and a transfer cap, illustrating the flow of disinfectant, according to an example embodiment of the present disclosure.

FIG. 3 illustrates a perspective view of a transfer set with integrated disinfectant chamber, with a transfer cap removed, and when mated to a connector during a fluid transfer, according to an example embodiment of the present disclosure.

FIGS. 4A-4B illustrate perspective views of a releasable connector with and without a pull ring attached, according to an example embodiment of the present disclosure.

FIGS. 5A-5B illustrate perspective views of a releasable connector before and after connecting to a transfer set with integrated disinfectant chamber, according to an example embodiment of the present disclosure.

FIGS. 6A-6B illustrate perspective views of a releasable connector before and after disconnecting to a transfer set with integrated disinfectant chamber, according to an example embodiment of the present disclosure.

FIG. 7 illustrates perspective view of a transfer set with integrated disinfectant chamber, with a transfer cap removed, and when mated to a releasable connector, according to an example embodiment of the present disclosure.

### DETAILED DESCRIPTION

Example embodiments will now be described more fully with reference to the accompanying drawings.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specific the presence of stated features, integers, steps, operations, elements, and/or components, but do not preclude the presence or additional of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent”). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

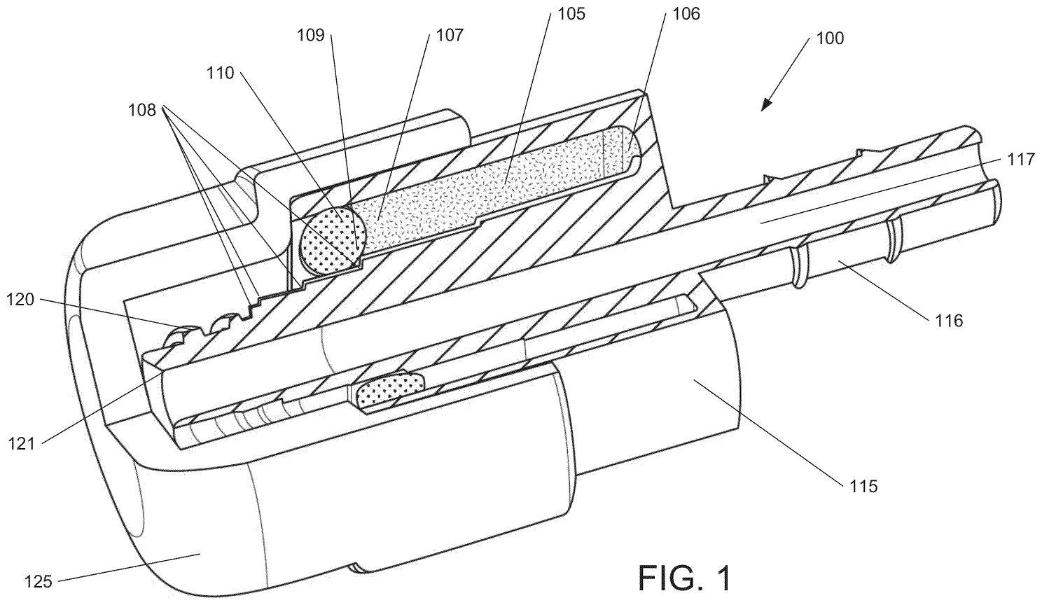

FIG. 1 illustrates a perspective view of a transfer set with an integrated disinfectant chamber and a transfer cap. Generally, the transfer set 100 includes a main body 115, which has a hollow tubular shape, and a transfer cap 125 removably disposed on a distal end of main body 115. The main body 115 has a catheter end 116 at its proximal end and a connecting end 121 at its distal end. Connecting end 121 further includes threads 120. The main body 114 includes a main opening 117 that provides a flow pathway from the connecting end 121 to the catheter end 116. The catheter end 116 is generally configured to be removably coupled with tubing, such as intravenous tubing and/or dialysis fluid set tubing (not shown) that may be subsequently connected to a user catheter.

At the connecting end 121, the user can place the transfer cap 125 so as to prevent undesired access to main body 115 and, more particularly, main opening 117. The main body 115 includes a disinfectant chamber 105 that is filled with a liquid or vapor disinfectant under pressure. In an example embodiment, the disinfectant is elemental iodine. However, alternative example embodiments can include, but are not limited to, disinfectants that are alcohol-based comparable to elemental iodine.

As illustrated, the disinfectant chamber 105 is a chamber formed around the circumference of the main body 115, so as to flank the exterior of the main body 115 without blocking the main opening 117. The disinfectant chamber 105 is generally disposed at a location between the catheter end 116 and the connecting end 121. Advantageously, the disinfectant chamber 105 is disposed as an integral component of transfer set 100, such that it is physically coupled to catheter end 116 and related main opening 117. In a particular example, the entire transfer set 100 with main body 115 and disinfectant chamber 105 is of single-piece construction, such as via injection-molding, blow-molding, or the like.

The disinfectant chamber 105 is located after the threads 120 and before the catheter end 116. The disinfectant chamber 105 has a closed end 106 adjacent to the catheter end 116 and an open end 107 adjacent to the connecting end 121. In addition, the main body includes a plurality of steps 108 between the threads 120 and the disinfectant chamber 105. As a result, the main body 115 has a stepped geometry that tapers outward between the threads 120 and the open end 107 of the disinfectant chamber 105. Namely, the diameter of the connecting end 121 adjacent to the disinfectant chamber 105 is greater than the diameter of the connecting end 121 adjacent to the threads 120.

The open end 107 of disinfectant chamber 105 is selectively sealable with an impermeable, non-porous barrier 110. In one embodiment, the barrier 110 is an O-ring made of a non-porous material, like rubber, a fluoro compound, or a fluoro polymer. Alternative embodiments can include a different barrier 110 that is impermeable. The barrier 110 is disposed within disinfectant chamber 105 adjacent to open end 107, such that it occupies the space between the open end 107 of the disinfectant chamber 105 and the step 108 closest to disinfectant chamber 105, and a gap 109 remains between the barrier and the step 108 closest to the disinfectant chamber. In turn, the barrier 110 selectively seals the open end 107 of the disinfectant chamber 105 so as to retain the disinfectant in the disinfectant chamber 105.

Notably, because the barrier 110 is impermeable and non-porous, the disinfectant diffuses out of the disinfectant chamber 105 at the gap 109. Specifically, vapor pressure to drives the disinfectant, through past the barrier 110 and out of the disinfectant chamber 105 through the gap 109. The disinfectant flows down the steps 108, towards the connection end 121 and subsequently coats the threads 120. It should be appreciated that the rate of flow of disinfectant (e.g., from chamber 105 toward threads 120) is optimized to ensure adequate sterilization while simultaneously conserving disinfectant to ensure maximum usable life of the disinfectant within chamber 105. It should be appreciated that this rate of flow of disinfectant can be optimized via material selection (e.g., material composition of barrier 110), disinfectant selection (e.g., type of disinfectant and/or pressure of disinfectant within disinfectant chamber 105), or the like.

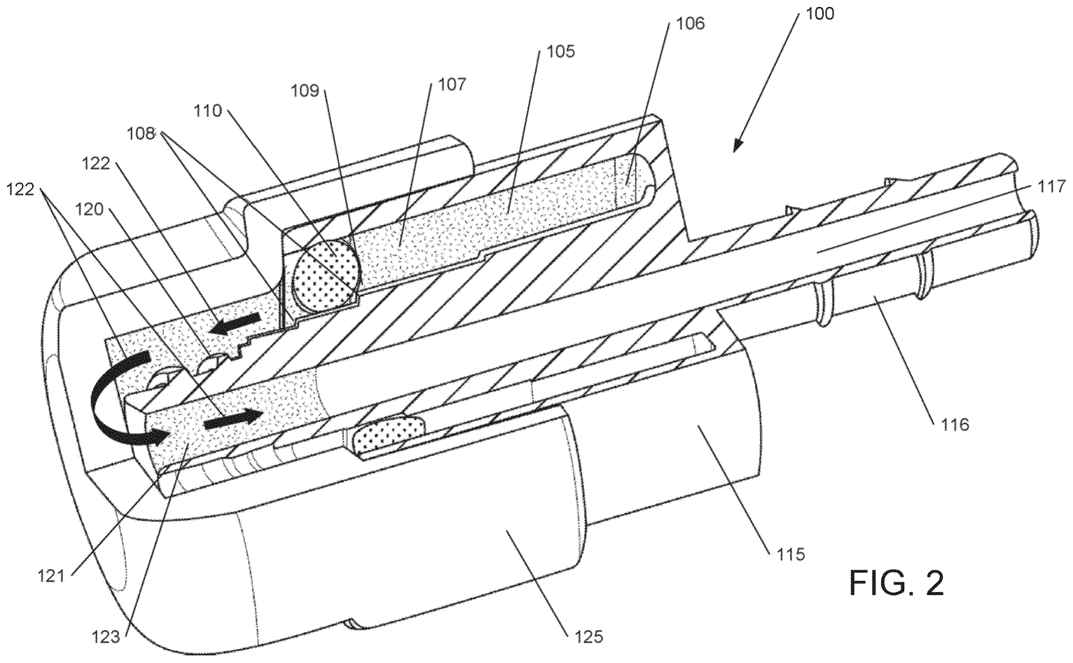

Continuing on, FIG. 2 illustrates the flow of disinfectant. Specifically, the flow direction arrows 122 demonstrate the path of the disinfectant: from the disinfectant chamber 105, past the barrier 110 through the gap 109, down the steps 108, onto the threads 120, and against the transfer cap 125 internal surface. In this embodiment, as illustrated, the disinfectant also flows to the inside 123 of the connecting end 121. As shown by FIG. 2, the disinfectant coats the surface of the threads 120 and the inside 123 of the connecting end 121, thereby disinfecting the threads 120 and inside 123 of the connecting end 121. The transfer cap 125 ensures that disinfectant remains in a “closed environment” and does not escape beyond the connecting end 121; rather, the disinfectant remains proximate to threads 120 and inside of the connecting end 123 so as to ensure optimum disinfection (e.g., during storage or transfer).

In an alternative embodiment, not shown, the transfer cap 125 includes an internal core that mates with the opening at the connecting end 121 when the transfer cap 125 is placed on the transfer set 100. In this alternative embodiment, the internal core on the transfer cap 125 seals and isolates the inside 123 of the connecting end 121 such that the disinfectant does not flow into or coat the inside 123 of the connecting end 121. The internal core can be made of the same material as, or a different material than, the transfer cap 125, so long as the internal core isolates the inside 123 of the connecting end 121. For example, in a variation of the alternative embodiment, the internal core is made of a deformable material, like rubber, that selectively deforms to insert into, and seal, the inside of the connecting end 121.

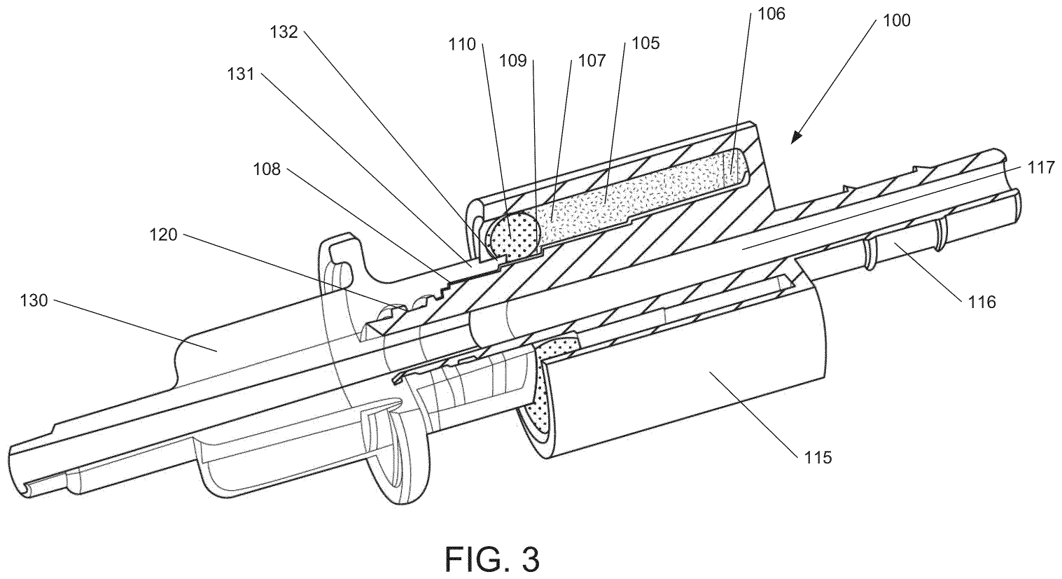

FIG. 3 illustrates a perspective view of a transfer set with integrated disinfectant when mated to a connector during a fluid transfer. As seen in FIG. 3, when the transfer set 100 is in use, the transfer cap 125 (not illustrated) is removed and a connector 130 mates with the main body 115 by interlocking with the threads 120 of transfer set 100. Specifically, the connector 130 creates a selectively sealed connection with the main body 115.

In this embodiment, a capping end 131 on the connector 130 engages with the barrier 110, such that the capping end 131 selectively seals the disinfectant chamber 105 and stops disinfectant diffusion. Specifically, the capping end 131 includes a diffusion stopper 132 that engages with the barrier 110 at, or around, the gap 109. The diffusion stopper 132 prevents vapor pressure from diffusing the disinfectant out of the disinfectant chamber 105, such that the disinfectant does not flow out of the disinfectant chamber 105 when the connector 130 is connected with the main body 115 while the transfer set 100 is in use, such as during peritoneal dialysis fluid transfer.

The composition and geometry of the diffusion stopper 132 can vary in different embodiments. For example, the diffusion stopper 132 can be made of the same material as the connector 130 and simply be the end of the capping end 131 configured to engage with the barrier 110 around the gap 109. Alternatively, the diffusion stopper 132 can be made of a selectively deformable material, like rubber or another medical-grade plastic, such that when the connector 130 connects to the main body 115, the diffusion stopper 132 envelopes the entirety of the barrier 110.

Importantly, during transfers, the connector 130 and related capping end 131 prevents the transfer set 100 from dripping or spilling disinfectant. Overall, this configuration ensures that the transfer set 100 only permits disinfectant to escape from disinfectant chamber 105 to disinfect threads 122 when transfer set 100 is not “in use” (i.e., when transfer set 100 is not coupled to a connector 130 for fluid transfer via main opening 117).

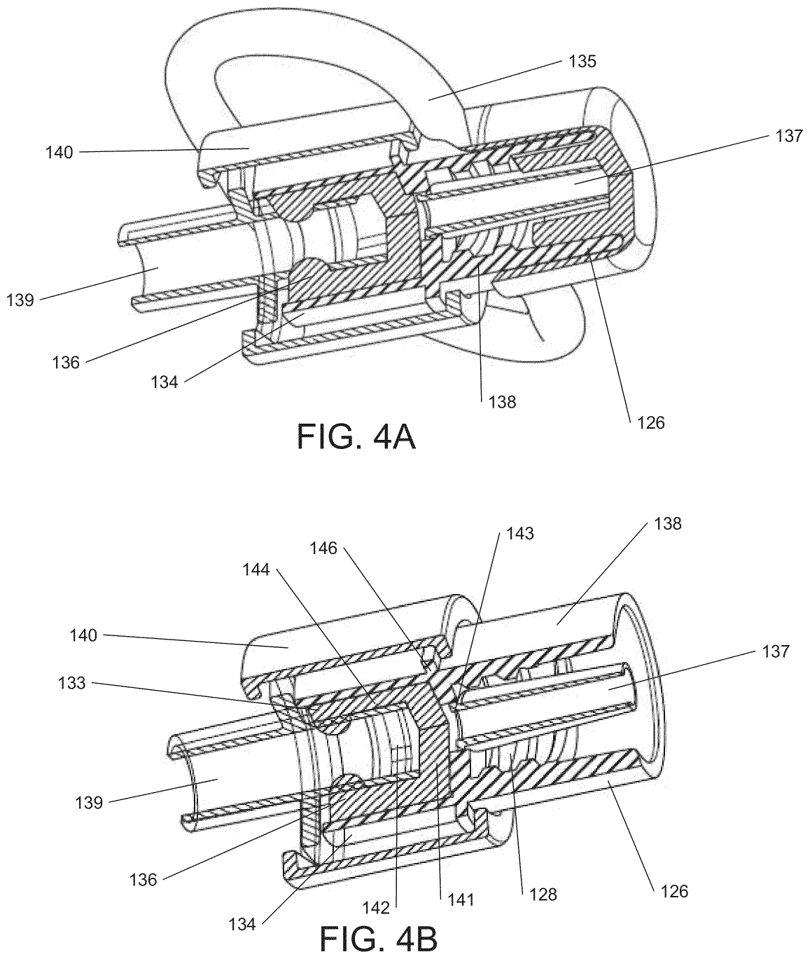

FIGS. 4A-4B illustrate perspective views of a releasable connector with and without a pull ring attached. The releasable connector 138 in FIGS. 4A-4B allows the user to connect in and out on of the main body 115 via a releasable connector 138 that selectively couples to the main body 115 in substitute of a transfer cap.

FIG. 4A shows the releasable connector 138 before use. As shown in FIG. 4A, when new, the releasable connector 138 includes a pull ring 135 selectively coupled to a first end 126 of the releasable connector 138. The pull ring 135 can be made of a selectively deformable material, such as rubber, to seal the first end 126 when coupled to the releasable connector 138. However, the deformable material of the pull ring 135 allows the user to grip the pull ring 135 and remove it from the first end 126 by pulling the pull ring 135 away from the first end 126. The releasable connector 138 further includes a second end 134, a septum 136, and a luer 137. A connector cap 139 is selectively coupled to the releasable connector 138 via a collar.

FIG. 4B illustrates the releasable connector 138 once the user removes the pull ring 135 and before connecting the releasable connector 138 to the main body 115. As seen in FIG. 4B, the septum 136 is located within the releasable connector 138 at the second end 134. The septum 136 includes an outer lip 133 that leads to an internal chamber 144, followed by an inner lip 141 that leads to a main body entry 146, which leads to a luer member 143. The septum 136 is made of a selectively deformable material, such as rubber or another medical-grade plastic. The septum 136 is fixedly coupled to an internal portion of the second end 134 of the releasable connector 138. Specifically outer lip 133 faces externally, away from the first end 126, and the inner lip 141 faces internally and is flush with a main body entry 146. As a result, the septum 136 is positioned inside the second end 134 of the releasable connector 138 with the outer lip 133 being externally facing and leading to the internal chamber 144, followed by the inner lip 141. The inner lip 141 covers the main body entry 146, thereby preventing unintended access and contamination, to maintain a sterile main body 115 environment.

As also seen in FIG. 4B, the connector cap 139 mates with the septum 136 such that the connector cap 139 partially occupies the internal chamber 144, with a cap groove 142 flush with the inner lip 141. The collar 140 secures the connector cap 139 within the releasable connector 138. In this embodiment, the collar 140 is made of a selectively deformable material that holds its shape, like a hard plastic, such that once the collar 140 is placed around the connector cap 139 and the releasable connector 138, the collar 140 retains its shape to keep the connection between the connector cap 139 and the releasable connector 138.

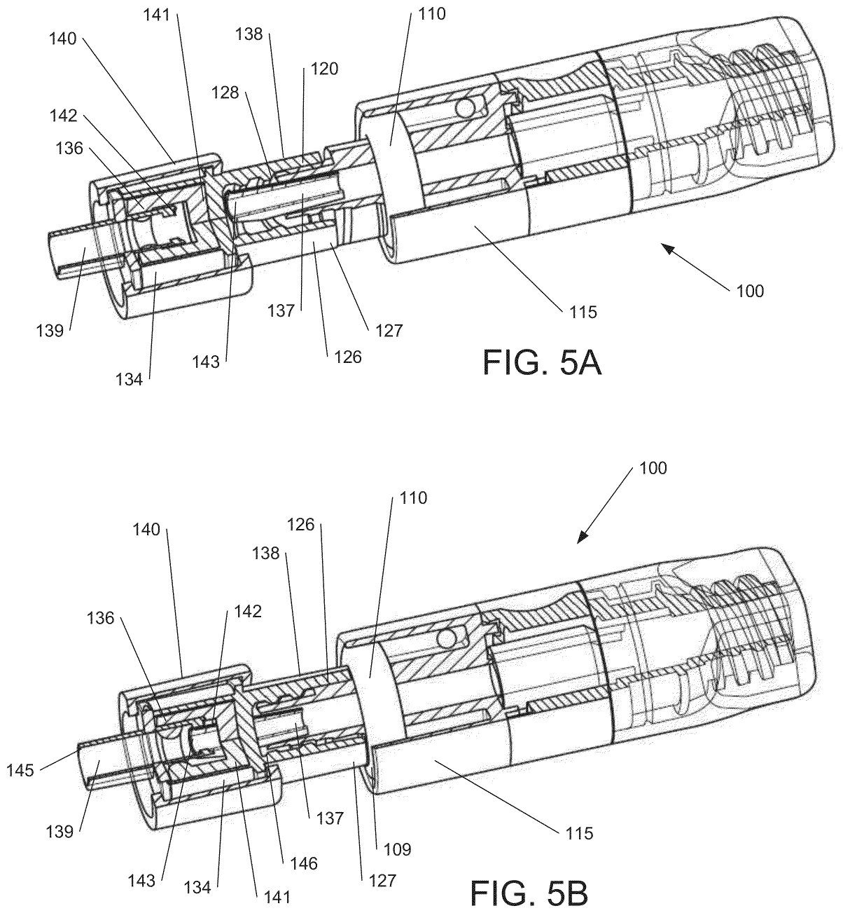

Next, FIGS. 5A-5B illustrate perspective views of a releasable connector before and after connecting to a transfer set with integrated disinfectant chamber. FIG. 5A shows the releasable connector 138 during connection to the main body 115. The first end 126 of the releasable connector 138 mates with the main body 115. Specifically, mating threads 128 on the internal surface of the first end 126 engage with the threads 120 from the main body 115 such that the user can rotate the releasable connector 138 relative to the main body 115, so as to selectively couple the releasable connector 138 with the main body 115 in a sealed connection. As the user does this, the luer 137 engages with the connecting end 121 of the main body 115, such that the luer 137 remains in place while the remainder of the releasable connector 138 moves towards the main body 115. It should be noted that the luer 137 is made of a hard material that retains its shape under stress, such as a hard medical-grade plastic. Moreover, the luer 137 has a hollow tube shape with a slight taper that enables it to fit only partially inside the connecting end 121, such that it remains in place while the rest of the releasable connector 138 moves closer to the main body 115.

FIG. 5B shows the releasable connector 138 when fully connected to the main body 115. The releasable connecter 138 is fully connected to the main body 115 once there is no space between the first end 126 and the barrier 110. In this embodiment, this coincides with the mating threads 128 fully engaging with the threads 120 from the main body 115, such that, for example, the user can no longer rotate the releasable connector 138 relative to the main body 115. This also coincides with the luer member 143 pushing past the main entry 135 and through the inner lip 141. Once past the inner lip 141, the luer member 143 couples with the cap groove 142. In this embodiment, the luer member 143 has a snap-fit relationship with the cap groove 142 that creates a sealed connection between the two. In turn, when the releasable connector 138 fully connects to the main body 115, there is a direct, fluid coupling from the main body 115, through the luer 137, and past the connector cap 139 to a distal end 145.

A diffusion blocker 127 on the first end 126 engages with the barrier 110, such that the first end 126 selectively seals the disinfectant chamber 105 and stops disinfectant diffusion. Specifically, the diffusion blocker 127 that engages with the barrier 110 at, or around, the gap 109. Similar to other embodiments, the diffusion blocker 127 prevents vapor pressure from diffusing the disinfectant out of the disinfectant chamber 105, such that the disinfectant does not flow out of the disinfectant chamber 105 while the releasable connector 138 is connected with the main body 115.

The composition and geometry of the diffusion blocker 127 can vary in different embodiments. For example, the diffusion blocker 127 can be made of the same material as the releasable connector 138 and simply be the end of the first end 126 configured to engage with the barrier 110 around the gap 109. Alternatively, the diffusion blocker 127 can be made of a selectively deformable material, such that when the releasable connector 138 connects to the main body 115, the diffusion blocker 127 envelopes the entirety of the barrier 110.

During use, the user couples a therapy source (not shown) to the distal end 145 of the connector cap 139. The geometry of the distal end 145 of the connector cap 139 is configured such that the therapy source couples to the distal end 145 so as to create a sealed fluid connection between the therapy source and the connector cap 139. In turn, the user creates a sealed fluid connection between the therapy source and the main body 115 (with releasable connector 138 serving as a fluid intermediary).

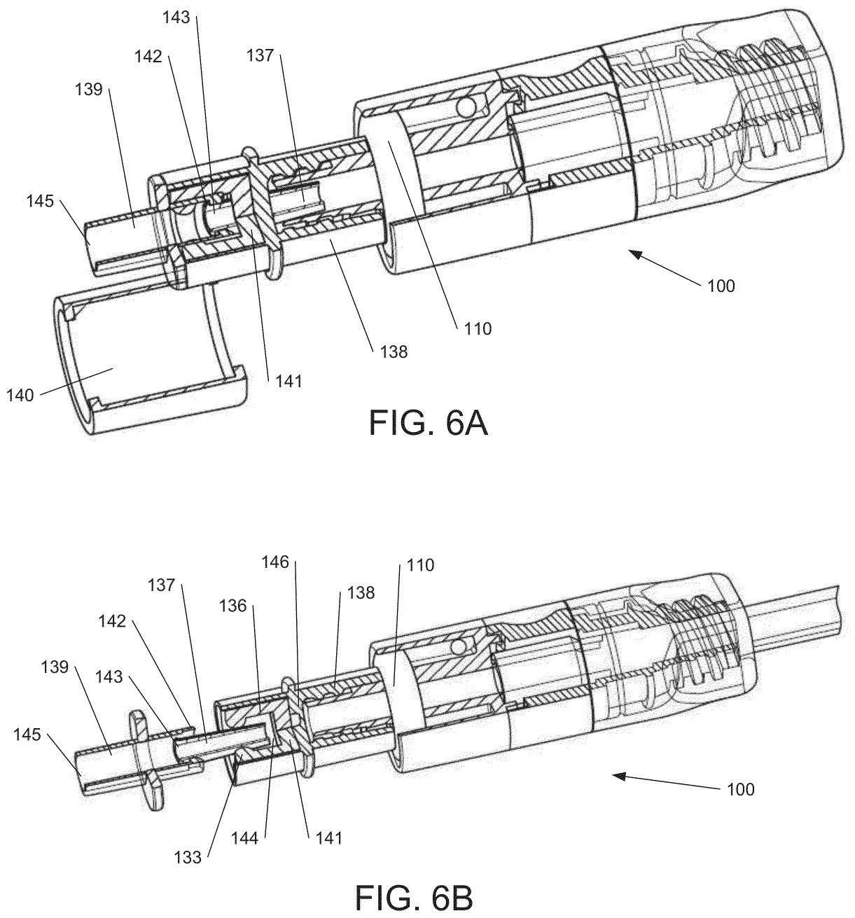

After completing therapy, the user removes the therapy source from the distal end 145. Next, the user removes the connector cap 139 from the releasable connector 138, as seen in FIG. 6A. As shown in FIG. 6A, the user first removes the collar 140. To achieve this, the user can pull the collar 140 to overcome the tension in the collar 140 and snap the collar 140 off the releasable connector 138. After this, as seen in FIG. 6B, the user removes the connector cap 139 from the septum 136 by pulling it away from the releasable connector 138. Because the luer 137 and connector cap 139 are coupled via the cap groove 142 and luer member 143, the luer 137 is also removed from the septum 136 when the user removes the connector cap 139. Notably, once the luer 137 disconnects from the main body entry 146 and moves away from the inner lip 141, the inner lip 141 returns to its original orientation, such that it entirely covers the main body entry 146. Finally, as the user pulls the luer 137 away from, and out of, the outer lip 133, the outer lip 133 returns to its original shape, at least partially covering access to the internal chamber 144. It should be noted that in other embodiments, the outer lip 133 completely covers access to the internal chamber 144.

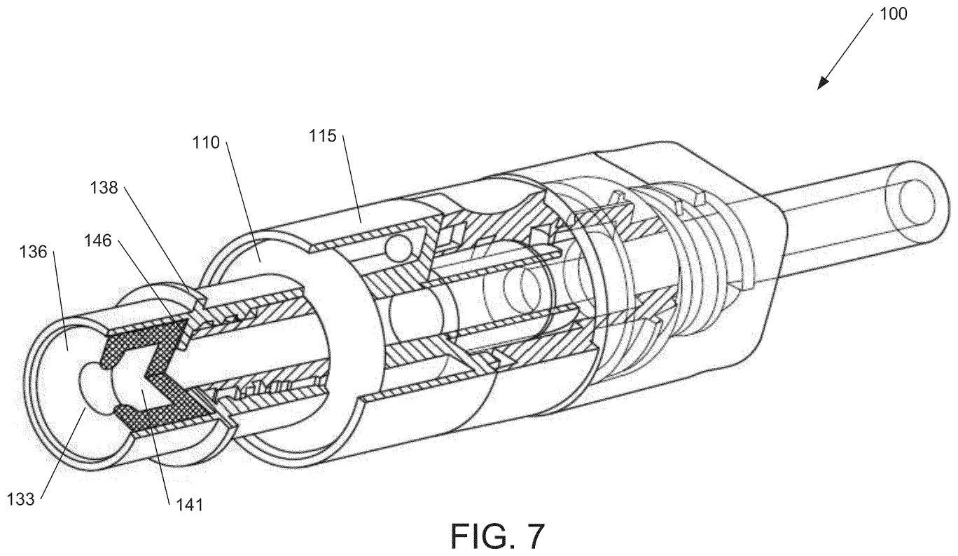

Finally, FIG. 7 shows a perspective view of the transfer set 100 when mated with the releasable connector 138 after the connector cap 139 is removed. The user can use the transfer set 100 by connecting a therapy source to the septum 136, past the outer lip 133, past the internal chamber 144, through the inner lip 141, and through the main body entry 146. As such, the user creates a sealed fluid connection between the therapy source and the main body 115 (with releasable connector 138 serving as a fluid intermediary). Overall, this allows the user to connect and disconnect to a therapy source without having to remove a transfer cap from a main body 115. In turn, by using the releasable connector 138, the user can maintain a sterile transfer set and prevent infections. Notably, the user is still able to remove the releasable connector 138 and place a transfer cap on the main body to allow for transfer set disinfection.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

1. A transfer set with integrated disinfectant, comprising:
a main body with a hollow tube shape, a catheter end, and a connecting end that includes mating components;
a cap configured to engage with the mating components; and
a disinfectant chamber integrated around an outer circumference of the main body;
wherein the catheter end selectively couples to tubing;
wherein the disinfectant chamber is located between the catheter end and the mating components;
wherein the disinfectant chamber includes a closed end adjacent the catheter end and an open end adjacent the mating components; and
wherein the disinfectant chamber contains a disinfectant.

2. The transfer set with integrated disinfectant of claim 1, further comprising a barrier located between the disinfectant chamber and the mating components, such that the barrier covers the open end and prevents the disinfectant from flowing towards the mating components.

3. The transfer set with integrated disinfectant of claim 2, wherein the barrier is comprised of an impermeable, non-porous material.

4. The transfer set with integrated disinfectant of claim 2, wherein the barrier is an O-ring.

5. The transfer set with integrated disinfectant of claim 1, wherein the disinfectant is elemental iodine.

6. The transfer set with integrated disinfectant of claim 1, wherein the disinfectant is an alcohol-based disinfectant.

7. The transfer set with integrated disinfectant of claim 1, wherein the barrier is comprised of one of a fluoro compound/fluoro polymer.

8. The transfer set with integrated disinfectant of claim 1, wherein the mating components are threaded.

9. The transfer set with integrated disinfectant of claim 2, wherein the main body further includes at least one notched portion around the outer circumference of the main body, such that when the barrier covers the open end, there is at least one gap between the barrier and the open end of the disinfectant chamber so as to permit flow of disinfectant through the distance between the barrier and the open end of the disinfectant chamber.

10. The transfer set with integrated disinfectant of claim 9, wherein a connector on a dialysis fluid bag obstructs the at least one gap between the barrier and the open end of the disinfectant chamber, such that the disinfectant remains in the disinfectant chamber.

11. The transfer set with integrated disinfectant of claim 9, wherein the cap further comprises a member configured to engage with an internal portion of the open end to seal the open end, such that the member prevents flow of the disinfectant.

12. A transfer set with integrated disinfectant, comprising:
a main body with a hollow tube shape, a catheter end, and a connecting end that includes mating components;
a releasable connector configured to engage with the mating components; and
a disinfectant chamber integrated around an outer circumference of the main body;
wherein the catheter end selectively couples to tubing and the connecting end selectively couples to the releasable connector;
wherein the releasable connector includes a first end configured to engage with the mating components and a second end with a resealable port;
wherein the disinfectant chamber is located between the catheter end and the mating components;
wherein the disinfectant chamber includes a closed end adjacent the catheter end and an open end adjacent the mating components; and
wherein the disinfectant chamber contains a disinfectant.

13. The transfer set with integrated disinfectant of claim 12, further comprising a barrier located between the disinfectant chamber and the mating components, such that the barrier covers the open end and prevents the disinfectant from flowing towards the mating components.

14. The transfer set with integrated disinfectant of claim 13, wherein the barrier is comprised of an impermeable, non-porous material.

15. The transfer set with integrated disinfectant of claim 12, wherein the disinfectant is one of elemental iodine or an alcohol-based disinfectant.

16. The transfer set with integrated disinfectant of claim 12, wherein the mating components are threaded.

17. The transfer set with integrated disinfectant of claim 13, wherein the main body further includes at least one notched portion around the outer circumference of the main body, such that when the barrier covers the open end, there is at least one gap between the barrier and the open end of the disinfectant chamber so as to permit flow of disinfectant through the distance between the barrier and the open end of the disinfectant chamber.

18. The transfer set with integrated disinfectant of claim 17, wherein the first end of the releasable connector obstructs the at least one gap between the barrier and the open end of the disinfectant chamber, such that the disinfectant remains in the disinfectant chamber.

19. The transfer set with integrated disinfectant of claim 18, wherein the resealable port on the second end of the releasable connector is configured to selectively fluidly couple to a connector on a dialysis fluid bag.

20. The transfer set with integrated disinfectant of claim 18, wherein the resealable port is configured to reseal after uncoupling to a connector on a dialysis bag.
